(19) United States
(12) Patent Application Publication
(10) Pub. No.: US 2026/0249136 A1
(43) Pub. Date: Aug. 27, 2026

(54) Real-Time Biometric Heart Rate Visualization for Competitive Sports Streaming

(71) Applicant: SENSAY LLC, New York, NY (US)

(72) Inventors: Raymond Lian, Brooklyn, NY (US); Nour Alddin Almasri, Brooklyn, NY (US); Justin Lynch, Oyster Bay, NY (US); Alex Ecklin, New York, NY (US)

(73) Assignee: SENSAY LLC, New York, NY (US)

(21) Appl. No.: 19/066,129

(22) Filed: Feb. 27, 2025

(51) Int. Cl. A63B 24/00; A63B 71/06; G16H 40/67

(52) U.S. Cl. CPC A63B 24/0062; A63B 71/0622; G16H 40/67; A63B 2071/065; A63B 2220/836; A63B 2225/20; A63B 2225/50; A63B 2230/06

(57) ABSTRACT

The present invention relates to a real-time biometric data visualization system for live sports streaming, integrating physiological performance metrics such as heart rate into live video broadcasts. The system comprises a wearable heart rate monitor, a mobile application for preprocessing biometric data, a backend server for storage and synchronization, and a web-based overlay system for generating dynamic graphical health bar visualizations. Biometric data is transmitted wirelessly via Bluetooth, Wi-Fi, or 5G from the wearable device to the mobile application, where it is filtered for noise and transmitted to the backend server at predefined intervals or when a significant change occurs. The overlay system retrieves this data in real time to render visual exertion levels synchronized with live video streams. This system enhances audience engagement, sports analytics, and coaching insights by providing instantaneous visibility into athlete exertion levels, enabling real-time strategic decision-making and immersive viewer experiences in competitive sports events.

### BACKGROUND OF THE INVENTION

### Field of the Invention

The present invention relates to real-time biometric data visualization in live video streaming platforms, specifically designed for competitive sports and high-intensity activities. This system enables the integration of athlete performance metrics, such as heart rate data, into live broadcasts, enhancing audience engagement, coaching insights, and sports analytics in real time.

### Problem Statement

Existing live streaming technologies lack real-time physiological data integration, preventing viewers, coaches, and analysts from gaining instantaneous insights into an athlete's exertion levels, endurance, and fatigue trends during competition. Current solutions primarily focus on post-event data review, limiting their usefulness in real-time performance assessment and strategic decision-making. Without live physiological feedback, audiences miss the ability to see and interpret an athlete's real-time physical effort, reducing engagement, analytical depth, and the effectiveness of in-game coaching adjustments.

In high-intensity sports, where real-time exertion monitoring could provide critical performance insights, the absence of seamless biometric integration results in missed opportunities for data-driven coaching, audience immersion, and improved athletic training methodologies.

### Prior Art and Existing Technologies

Several existing technologies attempt to incorporate biometric tracking and real-time performance monitoring, but they fall short in providing multi-athlete synchronized biometric visualization within live sports broadcasts.

### Biometric Data Synchronization With Video Streams

US20200126593A1 describes a method for synchronizing biometric data with recorded or live video streams. However, it focuses on post-event analysis and timestamp-based synchronization rather than real-time biometric overlays for multiple athletes in a competitive setting. Additionally, it lacks dynamically adjusting health bar visualizations based on exertion levels.

### Wearable Heart Rate Monitors & Fitness Trackers

U.S. Pat. No. 8,945,017B2 discloses a wearable heart rate monitor utilizing photoplethysmography (PPG) sensors for fitness tracking. However, it is designed for individual users rather than multi-athlete real-time streaming, and does not provide real-time low-latency data transmission for live sports broadcasts.

US20120116853A1 discusses systems for tracking biometric data across multiple athletes but focuses on data collection and transmission, lacking real-time integration into live video feeds with graphical overlays.

### Sports Telemetry & Tracking Systems

U.S. Pat. No. 8,289,185B2 introduces a telemetry system that collects real-time performance metrics and overlays them onto live video telecasts. However, it primarily tracks motion and positional data rather than physiological metrics such as heart rate and exertion level.

U.S. Pat. No. 11,902,603 covers embedding live tracking data into sports video streams, synchronized frame-by-frame, but focuses on positional data and predictive modeling, rather than real-time biometric visualization.

### Dynamic Overlay Systems & Secure Data Transmission

US20210168416A1 describes server-side manipulation of live video streams to insert advertisements dynamically. While relevant to real-time overlay insertion, this patent does not focus on biometric data or athlete tracking.

US20210192899A1 discusses secure biometric data transmission for live sports wagering. However, its focus is on betting analytics, rather than real-time athlete performance visualization.

### Transition to the Present Invention

The present invention overcomes the limitations of prior art by integrating real-time, multi-athlete biometric tracking into live video streams using dynamic health bar overlays. Unlike existing systems, it:
1. Enables low-latency, real-time data transmission through WebSocket/UDP protocols.
2. Uses match-based synchronization to track multiple athletes concurrently.
3. Implements dynamic graphical overlays that adjust based on real-time heart rate fluctuations and exertion levels.
4. Provides seamless integration with third-party streaming and analytics platforms for live sports events.

### SUMMARY OF THE INVENTION

The present invention is a real-time biometric heart rate visualization system designed for competitive sports streaming, integrating real-time physiological data into live video feeds. This system enables continuous tracking and visualization of athlete performance metrics, enhancing audience engagement, coaching insights, and sports analytics by delivering live biometric insights synchronized with the broadcast.

The system comprises a wearable heart rate monitor, a mobile application, a backend server, and a web-based overlay system. The wearable heart rate monitor captures biometric data and transmits it via Bluetooth, Wi-Fi, or 5G. The mobile application receives and pre-processes the incoming data, filtering noise, verifying changes in heart rate, and determining if an update is needed. It only transmits data to the backend when a heart rate change occurs or at five-second intervals, whichever comes first. The backend server is responsible for storing structured data, including each athlete's current heart rate, timestamps, maximum heart rate, and resting heart rate, within a structured fight document for each match. The web-based overlay system retrieves this stored data using HTTPS and renders real-time graphical overlays that visually represent exertion levels and biometric trends on live video streams.

Unlike existing solutions that focus on post-event analysis or non-competitive fitness tracking, this invention introduces a match-based synchronization system that allows multiple athletes to stream and compare biometric data simultaneously while maintaining a low-latency, synchronized experience across live broadcasts. By leveraging real-time biometric tracking, dynamic health bar representation, and seamless live video integration, this system eliminates delays, enhances live-streaming sports analytics, and provides an interactive and data-driven experience for viewers, coaches, and analysts.

This system bridges the gap between real-time sports analytics and live streaming technologies, making sports broadcasts, coaching, and performance analysis more immersive, efficient, and insightful.

### DETAILED DESCRIPTION

### Overview

The present invention is a real-time biometric heart rate visualization system designed for live sports streaming and competitive performance tracking. It enables instantaneous physiological data integration into live video feeds, providing audiences, analysts, coaches, and athletes with a dynamic, real-time view of exertion levels. Unlike existing solutions that focus on post-event data review, this invention delivers live biometric insights synchronized with the broadcast.

The system consists of four core components:
1. Wearable Heart Rate Monitor—Captures real-time heart rate data.
2. Mobile Application—Handles device pairing, pre-processing, and data transmission.
3. Backend Server—Stores and syncs biometric data for visualization.
4. Web-Based Overlay System—Displays biometric data as dynamic health bars overlaid on live video feeds.
By eliminating latency, ensuring secure data synchronization, and delivering real-time analytics, this system revolutionizes how biometric data is used in sports, gaming, and fitness applications.

### System Components and Architecture

The system is structured into multiple interconnected components, each serving a critical function in collecting, processing, and displaying real-time biometric data.

### Wearable Heart Rate Monitor

The wearable device is a biometric sensor-equipped monitor optimized for sports applications.
Sensor Technology: Utilizes Photoplethysmography (PPG) or Electrocardiography (ECG) sensors for heart rate detection.
Wireless Connectivity: Supports Bluetooth 5.0, Wi-Fi, or 5G for high-speed, low-latency transmission.
Form Factor: Available as a wristband, chest strap, armband, finger ring, or mouthguard, ensuring minimal interference with movement while providing accurate biometric readings.
Battery Optimization: Designed for extended use with low-power data transmission.

The wearable continuously transmits heart rate readings, ensuring high-frequency data sampling for real-time processing.

### Mobile Application

The mobile application serves as a centralized data relay hub, facilitating device pairing, biometric data pre-processing, and real-time visualization across multiple users and connected wearable devices. The application is optimized for low-latency, high-speed connectivity, ensuring continuous and accurate data transmission for live performance tracking.
Device Pairing & Authentication: Securely connects with wearable monitors via Bluetooth, Wi-Fi, or 5G, ensuring seamless authentication for each participant.
Match Creation & Synchronization: Generates unique match IDs, enabling multi-athlete biometric data streaming within a shared session.
Pre-Processing & Filtering: The mobile app removes noise, verifies heart rate changes, and determines if an update is required before sending data to the backend.
Data Transmission: The application transmits processed biometric data to the backend server only when the heart rate changes or every 5 seconds, whichever occurs first.
User Interface (UI): Displays real-time heart rate values, exertion levels, and recovery metrics. Allows customization for athletes, coaches, and analysts.
Multi-Device Compatibility: Supports smartwatches, fitness rings, and AI-enhanced biometric tracking devices, ensuring expanded data collection points for a comprehensive physiological assessment.
Offline Mode & Cloud Backup: Stores biometric data locally when connectivity is lost and synchronizes with the backend once restored.

The mobile application is engineered for multi-user synchronization, allowing for comprehensive biometric tracking in competitive settings while delivering instantaneous physiological feedback for real-time decision-making.

### Backend Server

The backend server serves as a secure data storage and retrieval hub, ensuring match-based synchronization and real-time biometric data availability for overlay processing. The backend does not perform exertion classification or filtering, as this is handled within the mobile application.

### Data Storage & Retrieval

The backend stores real-time biometric data for each match session, including:
Current heart rate
Timestamps
Resting heart rate
Max heart rate

Each athlete's biometric data is linked to a unique match ID, ensuring accurate synchronization across sessions. The backend only receives updates from the mobile application if the heart rate changes or if more than 5 seconds have passed since the last update.

### Exertion Level Processing & Performance Monitoring

The web-based overlay system retrieves match-specific biometric data from the backend via HTTPS requests.
The backend sends real-time updates to the overlay software whenever data is modified.
The overlay system calculates health bar values and renders exertion visualization based on:
   Resting heart rate (Full health)
   Max heart rate (Empty health)
   Intermediate health values determined by the number of available health bars.

### Latency Optimization & Performance Scaling

Real-Time Data Synchronization—The backend ensures instantaneous updates by responding to overlay system queries whenever data changes.
Session-Based Data Prioritization—Reduces polling frequency for low-exertion states while maintaining rapid updates for high-intensity actions.
Loss Prevention & Auto-Recovery—If a device disconnects, the backend retains the last known biometric values and seamlessly resumes tracking upon reconnection.

### Security & Compliance (Using Firebase Encryption Standards)

Data Encryption—The backend applies end-to-end encryption to secure all biometric transmissions.
Access Control—Uses authentication protocols to ensure only authorized clients retrieve match data.
Regulatory Compliance—Implements data privacy measures to protect athlete biometric data in competitive sports environments.

The backend server serves as the centralized repository for biometric data, enabling the overlay system to dynamically retrieve and render exertion-based visuals in real-time competitive sports streaming.

### Web-Based Overlay System (Real-Time Biometric Visualization)

The web-based overlay system retrieves match-specific biometric data from the backend server and renders graphical health bar overlays in real time. The overlay system does not perform any biometric processing or filtering; it only retrieves data and renders visual elements based on predefined health bar calculations.

### Overlay Data Retrieval & Synchronization

Retrieves match data from the backend server via HTTPS when a user initiates a session using a match ID.
Subscribes to data updates from the backend, ensuring that any changes in biometric data (HR updates every 5 seconds or upon HR change) are immediately reflected in the overlay.
Calculates visual health bar representation based on:
   Resting heart rate (Full health)
   Max heart rate (Empty health)
   Intermediate values determined by predefined health bar segmentation.

### Graphical Overlay Elements & Live Streaming Compatibility

Dynamically adjusts health bar segments based on live heart rate values.
Uses segmented graphical bars to represent exertion level in real time.
Displays real-time BPM values for each athlete, positioned adjacent to their health bar.
Supports side-by-side biometric comparison for multiple players in competitive matches.
Generates a browser-based overlay link compatible with OBS, Twitch, YouTube Live, and professional broadcasting software.

### Latency Optimization & Performance Scaling

Low-Latency Data Updates—Queries the backend server only when changes occur, reducing unnecessary polling.
Optimized Rendering—The overlay prioritizes recent biometric data, ensuring smooth visualization without buffering delays.
Scalability—Supports multi-athlete overlays with adjustable transparency and positioning controls.

### Security & Compliance (Using Firebase Encryption Standards)

Authenticated Data Access: Only authorized overlay sessions can request match-specific biometric data.
Data Protection: All transmitted biometric data is encrypted to ensure privacy compliance.

The web-based overlay system enables seamless integration of real-time biometric data into competitive sports streaming, ensuring accurate and synchronized health bar visualization for athletes, viewers, and analysts.

### Operation and Data Flow

### Match Creation & Device Pairing

1. User A creates a match in the mobile app, generating a unique match ID stored in the backend server.
2. Other players join the match using the match ID, enabling synchronized biometric tracking across multiple athletes.
3. Wearable monitors continuously measure biometric data (heart rate, HRV, etc.) and transmit the signal to the mobile app via Bluetooth, Wi-Fi, or 5G.
4. The mobile app handles pre-processing and filtering of biometric data, checking for heart rate changes or 5-second intervals before sending updates to the backend.
5. The mobile app transmits processed biometric data to the backend server only when necessary (HR change detected or 5-second threshold met).
6. The system enables real-time multi-athlete tracking, ensuring synchronized biometric data flow for competitors in the same match.

### Data Transmission & Processing

Wearable monitors continuously collect biometric data, including:
Heart Rate (HR)
Heart Rate Variability (HRV)
Oxygen Saturation (SpO2)
Exertion levels

The mobile app pre-processes biometric data before sending updates to the backend only when:
A heart rate change is detected, OR
A 5-second threshold is met.

The backend server stores match-specific biometric data, maintaining records for each competitor's:
Current heart rate (HR)
Timestamped HR data
Resting HR & Max HR values

The overlay system retrieves match data from the backend via HTTPS, ensuring real-time updates for heart rate visualization.

The web-based overlay system dynamically updates health bars, reflecting:
Full health at Resting HR
Empty health at max HR
Real-time exertion levels based on defined bar segment calculations.

### Live Streaming & Overlay Visualization

1. The web-based overlay system retrieves processed biometric data from the backend server using HTTPS requests, ensuring real-time synchronization with match data.
2. The overlay dynamically updates health bars, displaying:
   Full bars (low exertion)=Athlete at resting HR.
   Depleting bars (high exertion)=Athlete approaching max effort.
   Intermediate exertion levels dynamically calculated based on predefined bar segment values.
3. Graphical elements such as BPM counters and exertion indicators are updated in real time, reflecting fluctuations in heart rate and exertion status.
4. The overlay system allows customizable display settings, including:
   Adjustable transparency.
   Athlete-specific metric visualization.
   Multi-athlete comparative analysis.
   Supports seamless integration with professional broadcasting tools, including OBS, Twitch, YouTube Live, and other real-time streaming services.
5. Coaches and analysts can toggle between real-time and historical performance data, enabling:
   Enhanced in-match decision-making.
   Post-match performance reviews and analysis.

By expanding real-time data tracking, adaptive processing, and enhanced overlay visualization, this section ensures a seamless, low-latency, and immersive biometric streaming experience for athletes, coaches, and audiences alike.

### Alternative Embodiments

This invention is scalable and adaptable, allowing for future enhancements, including:
5G and Cloud-Based Streaming—Expanding range and reducing latency beyond Bluetooth/Wi-Fi limitations.
Customizable Visual Themes—Allowing organizations to brand overlays with team colors and custom UI elements.
Additional Biometric Sensors—Future expansion for oxygen saturation (SpO₂), skin temperature, and hydration tracking.
Integration with VR and Esports—Enabling biometric overlays for virtual reality sports training and esports competitions.

### Intended Use Cases

This invention is applicable across multiple industries, offering a transformative approach to real-time biometric visualization and performance tracking. The system's adaptability allows it to be utilized in competitive sports, training, medical applications, and immersive entertainment environments. Below are key industries that benefit from this technology:
1. Professional Sports Broadcasting—Enables real-time biometric tracking for endurance sports, MMA, Jiu-Jitsu, and team-based competitions.
   Live biometric overlays enhance audience engagement, allowing viewers to track athlete fatigue, exertion trends, and recovery metrics.
   Multi-athlete data comparisons support enhanced storytelling and strategic competition breakdowns.
2. Athlete Training & Performance Tracking—Provides coaches and trainers with live exertion data, optimizing workload distribution, injury prevention, and endurance tracking.
   Real-time biometric insights help customize training regimens based on individual exertion thresholds.
   Historical data synchronization enables long-term performance analysis for improvement tracking.
3. Esports & VR Training—Integrates biometric overlays into immersive gaming environments and VR-based training simulations.
   Competitive gamers benefit from real-time stress and fatigue tracking, optimizing reflex-based decision-making.
   VR simulations replicate high-intensity scenarios, improving reaction times and strategic adaptability.
4. Medical & Rehabilitation Monitoring—Supports cardiac recovery assessments, sports rehabilitation, and chronic condition tracking.
   Real-time biometric tracking helps therapists tailor recovery protocols based on exertion levels.
   Wearable biometric devices like mouthguards and rings track respiration and hydration levels, improving rehabilitation strategies.
5. Military & Tactical Training—Assists combat training and endurance drills through real-time biometric feedback.
   Soldier fatigue, stress levels, and exertion data allow for optimized training and enhanced decision-making under stress.
   Real-time tracking ensures physical conditioning remains within safety thresholds.
6. Corporate Wellness & Workplace Productivity-Enables organizations to integrate biometric monitoring into wellness programs.
   Heart rate variability and exertion tracking can reduce workplace stress-related fatigue.
   Real-time biometric insights help optimize work-life balance strategies.
7. Extreme Sports & Adventure Racing—Used in marathons, triathlons, ultra-endurance events, and adventure sports.
   Athletes and event organizers can monitor hydration levels, fatigue accumulation, and exertion intensity in real time.
   Race commentators leverage biometric overlays for real-time insights into athlete pacing and endurance.

By expanding real-time biometric tracking, adaptive processing, and enhanced visualization, this system delivers a comprehensive performance monitoring solution across multiple industries

### Key Advantages

This invention introduces unparalleled real-time biometric tracking for competitive sports, integrating advanced physiological monitoring with dynamic data visualization. The system's combination of low-latency data processing, real-time overlays, and multi-athlete tracking establishes a new industry standard for biometric applications in live sports and beyond.
1. Real-Time Audience Engagement—Viewers experience live exertion tracking, enhancing broadcast interactivity and creating a stronger connection with athletes.
   Spectators see and understand fatigue levels, recovery phases, and exertion peaks in real-time, transforming passive viewing into an interactive experience.
2. Live Performance Feedback for Coaches & Athletes—Enables instant physiological insights, allowing coaches to make real-time tactical adjustments based on athlete exertion levels.
   Crucial for combat sports, endurance races, and high-intensity competitions, where split-second decisions impact performance outcomes.
3. Seamless Integration with Existing Streaming & Training Tools—Fully compatible with OBS, YouTube Live, Twitch, and professional sports analytics platforms.
   Requires minimal setup and integrates directly into existing coaching, broadcasting, and performance analysis workflows.
4. Scalability & Multi-Athlete Support—Designed to track multiple competitors simultaneously, supporting team-based sports, dual-match competitions, and endurance events.
   The platform displays real-time biometric comparisons between athletes, enhancing strategic decision-making.
5. Low-Latency, High-Frequency Data Processing—Utilizes edge computing, real-time WebSocket/UDP protocols, and adaptive data prioritization to ensure:
   Continuous heart rate updates at millisecond accuracy, eliminating delays in exertion tracking and visualization.
6. Customizable & Expandable Data Visualization—Users can adjust biometric overlays, exertion thresholds, and alert parameters for different sports.
   Future expansion includes hydration tracking, blood oxygen levels, and stress monitoring, enhancing multi-metric performance tracking.
7. Application Beyond Competitive Sports—Optimized for sports but also viable for:
   Military training (live combat drills, endurance monitoring).
   Medical rehabilitation (real-time cardiac and respiratory tracking).
   Corporate wellness (monitoring stress and exertion in high-performance workplaces).
   Esports performance tracking (physiological tracking during competition).

By integrating cutting-edge biometric tracking, AI-driven analytics, and adaptive visualization, this system redefines performance monitoring, audience engagement, and live sports analytics.

### Conclusion

This real-time biometric heart rate visualization system is a breakthrough solution for competitive sports broadcasting, integrating physiological data into live sports coverage.

By introducing dynamic health bars, real-time exertion tracking, and seamless streaming integration, this system revolutionizes sports analytics and enhances viewer engagement.
The combination of low-latency biometric tracking, scalable multi-athlete monitoring, and adaptive overlays ensures broad applicability across competitive sports, training, medical rehabilitation, and immersive performance environments.

This invention sets a new benchmark for real-time sports analytics and biometric-driven audience engagement, making it a critical innovation for the future of live competitive sports visualization.

### BRIEF DESCRIPTION OF THE DRAWINGS

The following figures provide a visual representation of the real-time biometric heart rate visualization system for competitive sports streaming. These diagrams illustrate the system architecture, data processing flow, wearable hardware components, mobile application interface, and integration with broadcasting platforms.

The following figures provide a visual representation of the real-time biometric heart rate visualization system for competitive sports streaming. These diagrams illustrate the system architecture, data processing flow, wearable hardware components, mobile application interface, and integration with broadcasting platforms.

FIG. 1—System Overview Diagram

FIG. 1 illustrates the system architecture for real-time biometric data processing. The wearable heart rate monitor (101) sends processed HR updates to the mobile application (102), which determines when to transmit data to the backend server (103) based on HR changes or time intervals. The backend stores HR data, timestamps, and player-specific metrics for retrieval by the web-based overlay system (104), which generates real-time graphical overlays applied to the live video stream (105).

FIG. 2—Data Flow Diagram

FIG. 2 illustrates the data flow from raw biometric data input (201) to the real-time biometric overlay display (206). The mobile application (202) receives heart rate data from the wearable monitor, pre-processes and filters it (203), then transmits updates via WebSocket/UDP (209) to the backend server (204) for storage. The overlay system (205) retrieves this data using HTTPS (210), calculates exertion-based health bars, and renders the final graphical overlay (211) for display on the live video stream (206).

FIG. 3—Wearable Heart Rate Monitor Diagram

FIG. 3 depicts the internal components of the wearable heart rate monitor. It includes the outer casing (301), optical sensors and electrodes (302), microcontroller and processor (303), wireless communication module (304) for Bluetooth/Wi-Fi/5G transmission, and the battery and power management unit (305). The diagram shows the flow of data from sensor detection to wireless transmission (306).

FIG. 4—Mobile App UI Mockup

FIG. 4 provides a wireframe representation of the mobile application's user interface (UI). It illustrates the arena selection screen (401), player selection options (402), the choice to host or join a match (403), the heart rate monitor connection screen (404), and the match waiting/search interface (405). The final screen shows the overlay link generation & OBS integration option (406), which allows users to generate a browser source link for broadcasting software.

FIG. 5—Web-Based Overlay System Diagram

FIG. 5 shows how biometric data is displayed in real-time on a live video stream. The video feed (501) contains overlay elements such as heart rate overlay graphics (502), exertion health bars (503), and a 15-segment heart rate visualization (504). A BPM counter (505) is displayed next to each athlete's health bar, reflecting real-time exertion levels.

FIG. 6—OBS/Twitch/Youtube Overlay Integration

FIG. 6 presents the workflow for integrating the biometric overlay into streaming software (OBS, Twitch, YouTube, etc.). It starts with overlay link generation (601), which is copied & pasted into OBS/Twitch (602) as a browser source (603). The overlay is previewed (604), adjusted for size and positioning, and finally displayed on a live stream (605).

FIG. 7—Backend Data Processing & Cloud Storage

FIG. 7 illustrates the backend data processing and cloud storage workflow. The mobile application (701) transmits real-time heart rate (HR) data, match ID, and session details to the backend server (702) for storage and synchronization. The data update listener (703) continuously monitors HR changes and pushes real-time updates to connected applications. The overlay system API request (704) allows the overlay software to retrieve and sync match-specific HR data via HTTPS, ensuring live biometric visualization during competitive sports streaming.

FIG. 8—Alternative Embodiments/Future Enhancements

FIG. 8 showcases potential future expansions of the system, including third-party API integration (801) for external analytics, additional sensors (802) to track hydration and respiratory rates, and VR & esports integration (803) for immersive biometric tracking in gaming environments.

### DETAILED DESCRIPTION OF THE DRAWINGS

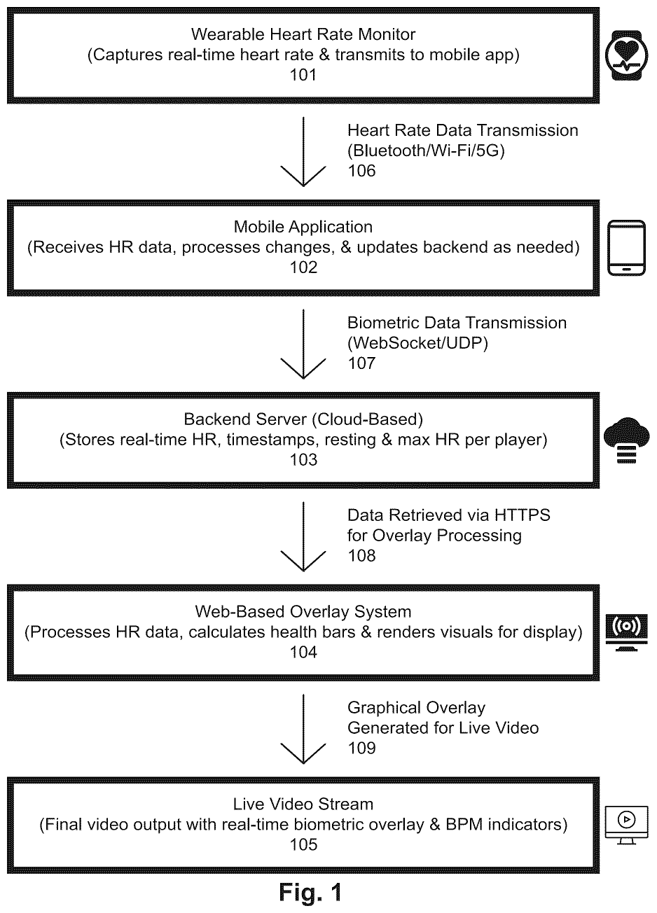

FIG. 1 illustrates the overall architecture of the real-time biometric heart rate visualization system, demonstrating how biometric data is captured, processed, transmitted, stored, and displayed as a live video overlay. The components are arranged in a top-down sequence, reflecting the flow of data throughout the system.

Wearable Heart Rate Monitor (101): Positioned at the top of the diagram, the wearable device (e.g., wristband, chest strap, finger ring, or mouthguard) detects real-time heart rate data using PPG or ECG sensors. The device continuously monitors biometric changes and processes the HR signal to determine if an update is needed.
Arrow 106 (Pointing Down): Represents processed HR data updates transmitted to the mobile application.

Mobile Application (102): Acts as the central hub for receiving and processing biometric data from the wearable device. The app verifies HR changes and ensures data transmission only when the HR value changes or after five seconds, whichever occurs first.
Arrow 107 (Pointing Down): Represents HR data transmission decision from the mobile app to the backend server.

Backend Server (103): A cloud-based database system that stores HR data, timestamps, resting/max HR values, and other session-based metrics. The backend ensures match-based synchronization, enabling real-time data retrieval for overlay generation.
Arrow 108 (Pointing Down): Represents data storage and retrieval functions in the backend.

Web-Based Overlay System (104): Responsible for retrieving stored HR data and rendering graphical overlays in real-time. The overlay system calculates exertion levels, updates health bar visuals, and integrates real-time BPM counters for live display.
Arrow 109 (Pointing Down): Represents processed graphical overlays sent to the video stream.

Live Video Stream (105): The final output, where the biometric overlay visuals (e.g., exertion health bars and BPM counters) are applied to live-streamed content. The system ensures low-latency synchronization, allowing viewers, coaches, and analysts to observe athlete performance in real time.

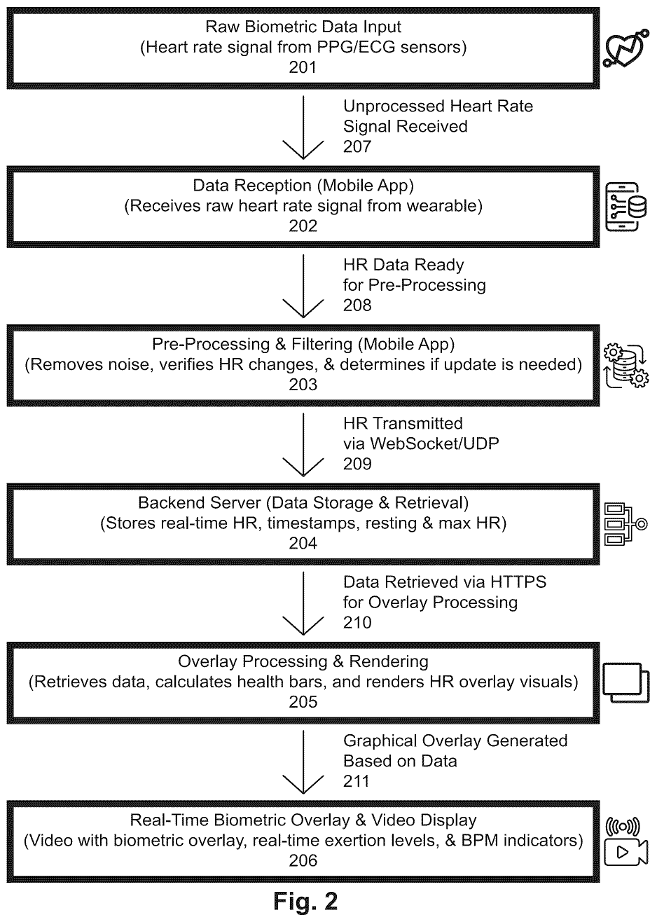

FIG. 2 illustrates the flow of biometric data from initial heart rate detection to the real-time biometric overlay visualization. The diagram follows a top-down approach, demonstrating how raw biometric data is received, processed, stored, and displayed.

Raw Biometric Data Input (201)—The wearable heart rate monitor captures real-time heart rate signals using PPG or ECG sensors and transmits them to the mobile application.
Arrow 207 (Pointing Down): Represents unprocessed heart rate signal being received by the mobile application.

Data Reception (Mobile App) (202)—The mobile app receives the heart rate signal from the wearable monitor and prepares it for processing.
Arrow 208 (Pointing Down): Represents raw heart rate data ready for pre-processing.

Pre-Processing & Filtering (Mobile App) (203)—The mobile app filters out noise, verifies if the heart rate has changed, and determines if an update is needed. Data is processed when either:
A heart rate change is detected, or
The last update occurred more than five seconds ago.
Arrow 209 (Pointing Down): Represents heart rate data transmission via WebSocket/UDP to the backend.

Backend Server (Data Storage & Retrieval) (204)—The backend stores and updates a fight document containing:
Current heart rate, timestamp, resting HR, and max HR for each player.
Arrow 210 (Pointing Down): Represents data retrieval via HTTPS for overlay processing.

Overlay Processing & Rendering (205)—The overlay system retrieves stored biometric data, calculates visual health bars, and prepares the graphical overlay using predefined rendering rules.
Arrow 211 (Pointing Down): Represents graphical overlay generation based on stored data.

Real-Time Biometric Overlay & Video Display (206)—The final output is a live-streamed video with a biometric overlay showing:
Heart rate fluctuations,
Exertion-based health bars, and
Real-time BPM indicators.

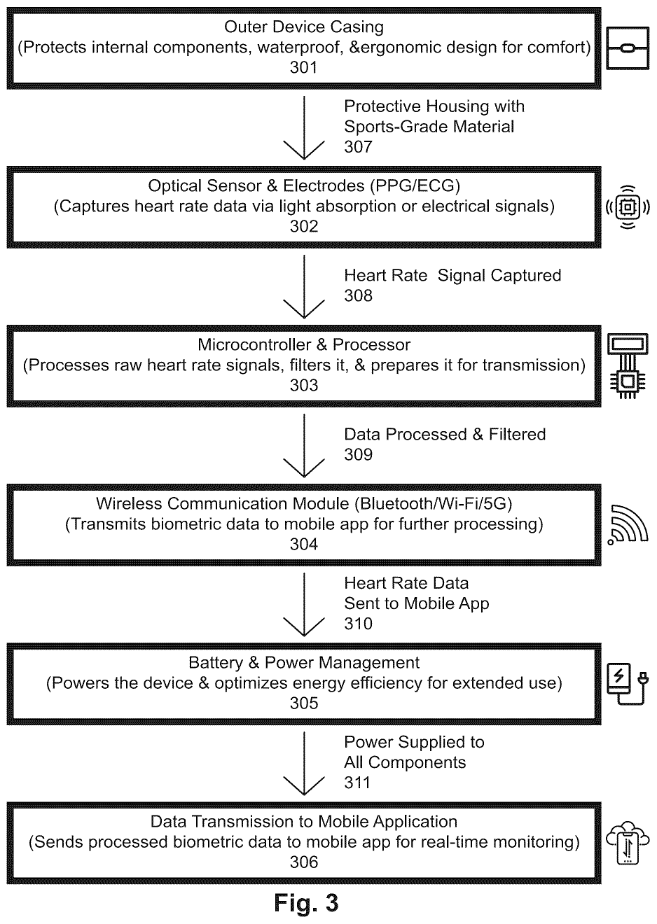

FIG. 3 provides a detailed breakdown of the wearable heart rate monitor and its internal components. It follows a top-down exploded view, showing how each part contributes to biometric data detection, processing, and transmission.

Outer Device Casing (301): The external structure of the device, made of sports-grade waterproof material, designed to protect internal components while ensuring comfort for athletes.
Arrow 307 (Pointing Down): Represents the protective role of the casing in shielding internal components.

Optical Sensor & Electrodes (PPG/ECG) (302): The heart rate detection module that captures biometric data using light absorption (PPG) or electrical signals (ECG).
Arrow 308 (Pointing Down): Represents heart rate signal detection and transmission to the processor.

Microcontroller & Processor (303): The processing unit that filters noise, extracts relevant biometric features, and converts heart rate signals into digital data for further processing.
Arrow 309 (Pointing Down): Represents data processing and preparation for transmission.

Wireless Communication Module (Bluetooth/Wi-Fi/5G) (304): Enables real-time transmission of biometric data to the mobile app via Bluetooth, Wi-Fi, or 5G connectivity.
Arrow 310 (Pointing Down): Represents data transmission to mobile devices.

Battery & Power Management (305): The power supply unit, ensuring long-lasting operation and energy efficiency.
Arrow 311 (Pointing Down): Represents power distribution across components.

Data Transmission to Mobile Application (306): The final step where the processed biometric data is sent wirelessly to the mobile app for real-time monitoring.

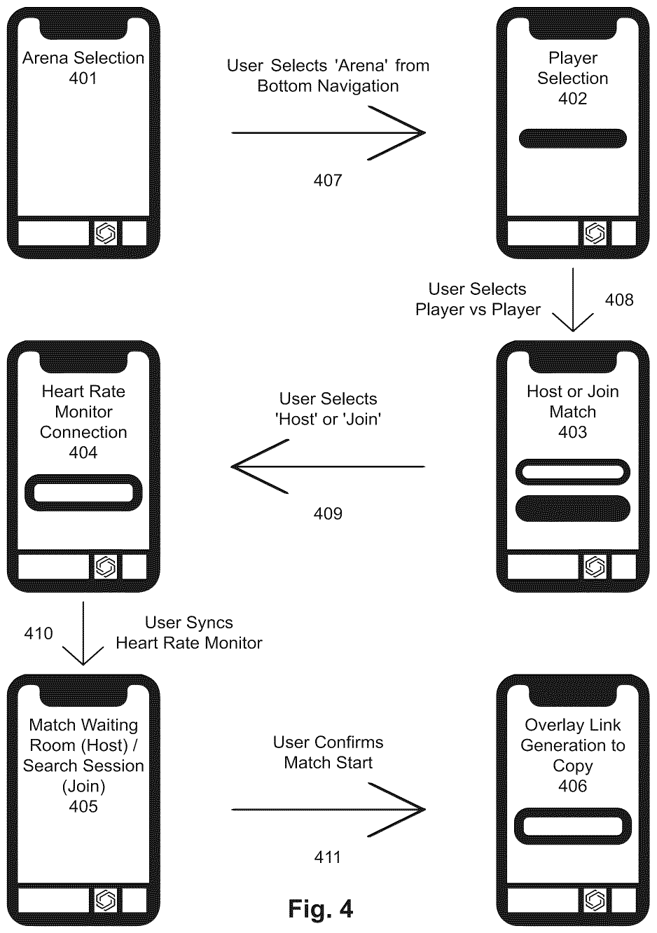

FIG. 4 illustrates the mobile application's user interface (UI) workflow, guiding users through match setup, device pairing, and overlay generation. The vertical sequence of screens represents a typical user journey.

Arena Selection (401): The first screen where the user selects “Arena” from the bottom navigation menu to begin match setup.
Arrow 407 (Pointing Down): Represents the transition to the Player Selection screen after tapping “Arena”.

Player Selection (402): The screen where users choose between different game modes, selecting “Player vs Player” for a head-to-head biometric tracking match.
Arrow 408 (Pointing Down): Represents navigation to the Host/Join Match screen.

Host or Join Match (403): The screen where users host a new match or join an existing session by searching for active players.
Arrow 409 (Pointing Down): Represents the transition to the Heart Rate Monitor Connection screen.

Heart Rate Monitor Connection (404): The screen prompts users to pair their heart rate monitor via Bluetooth before proceeding.
Arrow 410 (Pointing Down): Represents movement to the Match Waiting Room (Host) or Match Search (Join) screen.

Match Waiting Room (Host)/Search Session (Join) (405):
Host: Waits for a second player to connect.
Join: Searches for available sessions and selects a match to join.
Arrow 411 (Pointing Down): Represents the transition to Overlay Link Generation.

Overlay Link Generation & OBS Integration (406): Once both players are connected, the app generates a sharable link for OBS, Twitch, or YouTube to integrate the biometric overlay into a live stream.

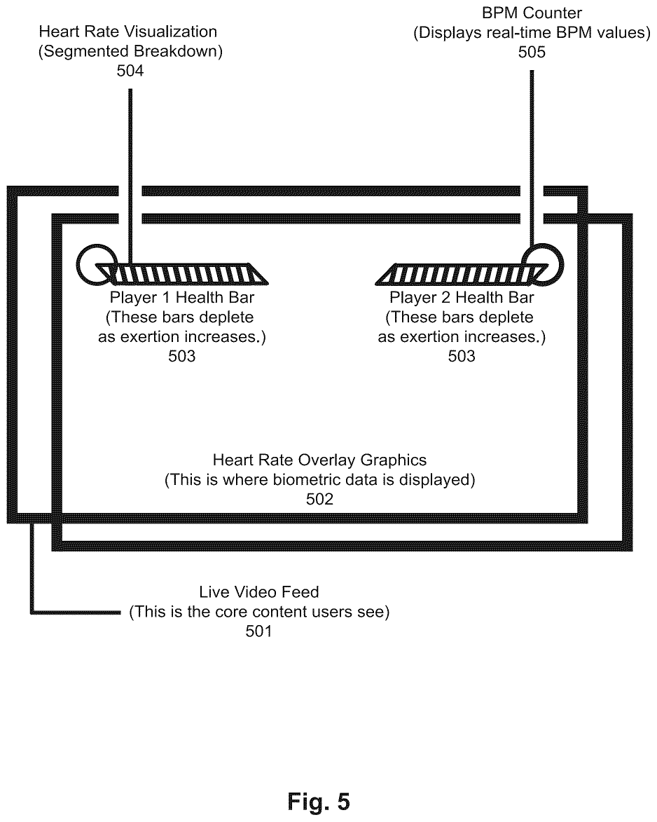

FIG. 5 illustrates how real-time biometric data is displayed over a live-streamed match, showing the graphical components of the overlay system.

Live Video Feed (501): The main background layer representing the live sports footage in which the biometric overlay is displayed.
This is the primary content visible to audiences.

Heart Rate Overlay Graphics (502): A transparent layer applied over the live video feed, displaying real-time biometric data.
This includes visual elements such as health bars and BPM counters.

Player Health Bars (503):
Left Side: “Player 1 Health Bar”
Right Side: “Player 2 Health Bar”
As exertion increases, bars visually deplete based on heart rate data.

Visual HR Segments (504)—Segmented Bar System:
Each health bar consists of smaller equal segments.
Segments dynamically adjust based on heart rate intensity.

BPM Counter (505):
Displays real-time BPM values next to each player's health bar.
This provides numerical verification of exertion levels.

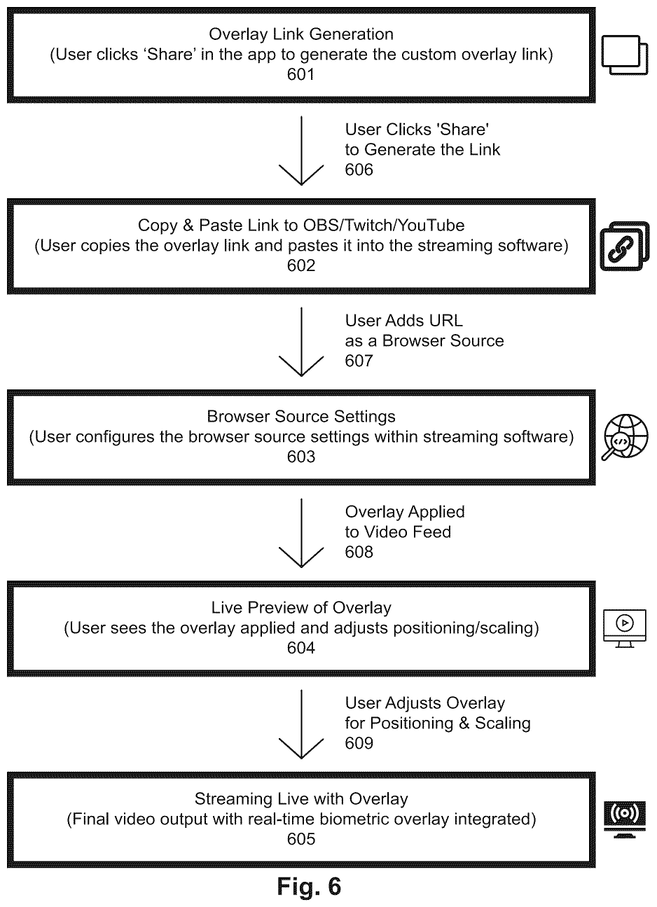

FIG. 6 illustrates the process of integrating the biometric overlay into third-party streaming platforms, such as OBS, Twitch, and YouTube Live. This figure demonstrates how the system generates a shareable overlay link, configures it as a browser source, and streams it with real-time biometric visuals.

Overlay Link Generation (601): The app generates a custom URL that hosts the real-time biometric overlay.
Arrow 606 (Pointing Down): Represents the user clicking ‘Share’ to copy the overlay link.

Copy & Paste Link to OBS/Twitch/YouTube (602): The user copies the overlay link and pastes it into OBS Studio, Twitch Studio, or YouTube Live.
Arrow 607 (Pointing Down): Represents the user adding the URL as a browser source.

Browser Source Settings (603): The user configures settings in OBS (or other platforms), such as positioning, transparency, and scaling.
Arrow 608 (Pointing Down): Represents the overlay applied to the video feed.

Live Preview of Overlay (604): The user sees a preview of the biometric overlay applied to the live video stream.
Arrow 609 (Pointing Down): Represents final positioning adjustments before going live.

Streaming Live with Overlay (605): The final broadcast with real-time biometric overlays is displayed to audiences.

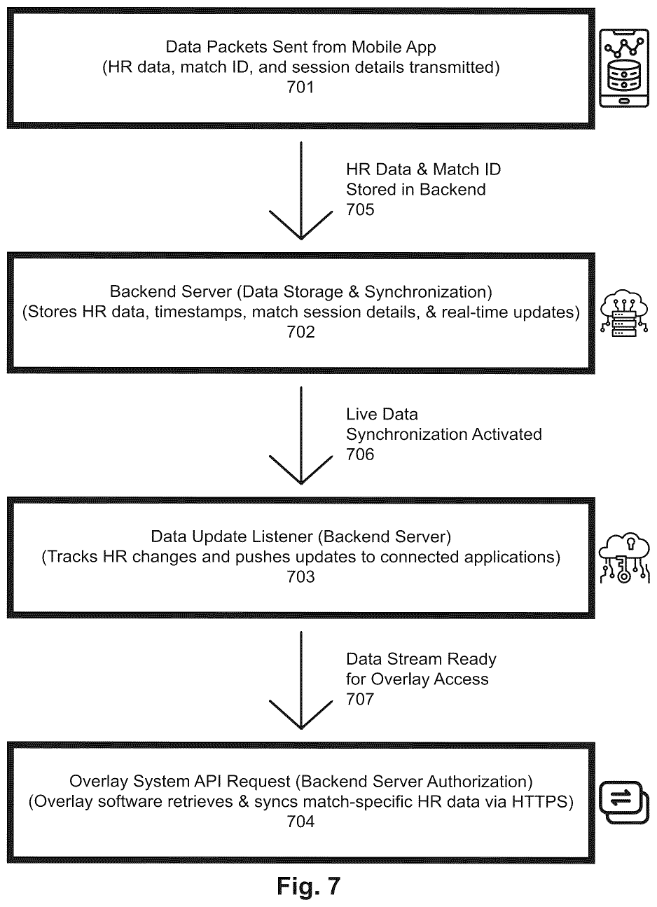

FIG. 7 illustrates the backend data pipeline responsible for storing, retrieving, and synchronizing real-time biometric data for live overlay visualization. The backend system ensures seamless data handling by managing heart rate data updates, match synchronization, and overlay processing.

Data Packets Sent from Mobile App (701): The mobile application transmits real-time biometric data, including heart rate readings, timestamps, and exertion metrics, to the backend server for processing. Data is sent when a heart rate change is detected or every 5 seconds, whichever occurs first.
Arrow 705 (Pointing Down): Represents the transmission of heart rate data from the mobile app to the backend server.

Backend Server (Data Storage & Synchronization) (702): The backend server stores incoming biometric data in structured fight documents, associating each data set with a unique match ID for accurate session tracking. The stored fight document contains real-time heart rate values, timestamps, resting heart rate, and max heart rate for each player.
Arrow 706 (Pointing Down): Represents secure storage of heart rate data and match-based synchronization.

Data Retrieval & Synchronization (703): The backend server is configured to handle real-time data requests and synchronization for active matches. When a user initiates an overlay session, the backend retrieves the latest biometric data for the associated match ID and continuously updates the front-end as new data is received.
Arrow 707 (Pointing Down): Represents real-time data retrieval and synchronization for active overlays.

Overlay Software Data Request & Sync (704): The overlay software, hosted separately from the backend, is authorized to access the stored fight document via HTTPS and a secure API. When the overlay software is launched using a shared match ID, it requests match-specific biometric data from the backend and establishes a connection to receive updates in real-time.
Arrow 708 (Pointing Down): Represents the overlay software querying the backend for match-based biometric data.

Real-Time Biometric Overlay Synchronization (705): Once the overlay software is authorized, the backend server continuously pushes updates whenever heart rate changes are detected or when the predefined time threshold is met. The overlay receives and processes the latest heart rate values to ensure an accurate live display.
Arrow 709 (Pointing Down): Represents continuous data synchronization between the backend and the overlay software.

This backend processing system ensures real-time biometric data accuracy, efficient match-based synchronization, and seamless communication between the mobile application, backend server, and overlay software for live sports visualization.

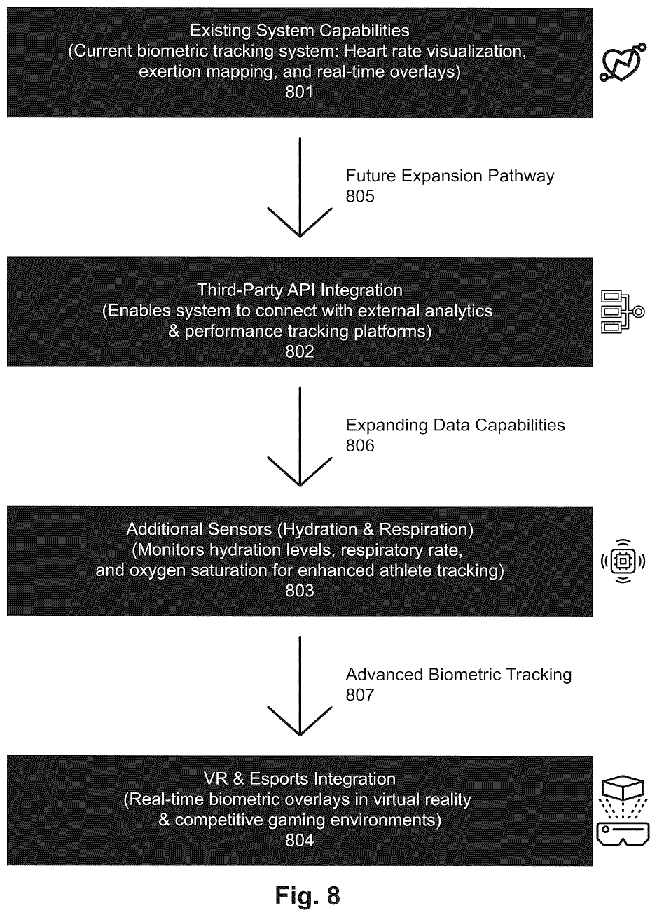

FIG. 8 outlines potential system upgrades, including third-party analytics, additional biometric sensors, and VR/esports integration.

Existing System Capabilities (800): Current system includes real-time heart rate tracking, exertion mapping, and biometric overlays.
Arrow 805 (Pointing Down): Represents expansion via third-party APIs.

Third-Party API Integration (801): The system can integrate with external analytics platforms, providing advanced data insights.
Arrow 806 (Pointing Down): Represents the addition of new biometric sensors.

Additional Sensors (802): Future iterations could track hydration levels, respiratory rate, and oxygen saturation for enhanced athlete monitoring.
Arrow 807 (Pointing Down): Represents expansion into VR and esports applications.

VR & Esports Integration (803): Real-time biometric overlays could be applied in virtual reality training and esports competitions, enabling biometric-enhanced gaming environments.

1. System for Real-Time Biometric Data Visualization
1. A system for real-time biometric data visualization in live sports streaming, comprising a wearable heart rate monitor configured to detect and wirelessly transmit real-time heart rate data via Bluetooth, Wi-Fi, or 5G, wherein the device continuously captures heart rate fluctuations and provides high-frequency sampling optimized for real-time processing; a mobile application configured to pair with the wearable monitor and authenticate users, preprocess biometric data by applying noise filtering and dynamic update logic based on heart rate fluctuations to ensure data is only transmitted when a significant change occurs or at predefined time intervals, and transmit only necessary biometric updates to reduce redundant data transmission and optimize real-time performance; a backend server configured to store and synchronize biometric data based on match sessions for multiple athletes, ensure real-time data access via a secure API to facilitate immediate retrieval for overlay synchronization, and process multi-athlete biometric data synchronization to enable synchronized visualization across all competitors in a given match session; and a web-based overlay system configured to retrieve real-time biometric data from the backend server via HTTPS, dynamically generate graphical health bar overlays representing athlete exertion levels in real time, and integrate seamlessly with third-party live streaming platforms for synchronized biometric data and video feed visualization.

2. The system of claim 1, further comprising a match-based synchronization module that synchronizes biometric data across multiple athletes in the same session to ensure real-time comparative performance visualization.

3. The system of claim 1, wherein the wearable heart rate monitor is in the form of a chest strap, armband, wristband, finger ring, or mouthguard, designed to capture biometric data with minimal interference to the athlete's movements.

4. The system of claim 1, wherein the mobile application transmits biometric data using WebSocket or UDP protocols to achieve ultra-low-latency data updates, thereby reducing network congestion and optimizing real-time transmission.

5. The system of claim 1, wherein the backend server ensures efficient data synchronization through session-based prioritization by reducing polling frequency for low-exertion states while maintaining high-frequency updates for intense exertion periods.

6. The system of claim 1, wherein the web-based overlay system dynamically adjusts the visualization of biometric data by recalibrating exertion thresholds based on real-time heart rate fluctuations and predefined athlete-specific exertion levels.

7. The system of claim 1, wherein the overlay system generates a browser-based interface for embedding biometric visualizations into live sports broadcasts, including support for integration with OBS, Twitch, YouTube Live, and other streaming platforms.

8. Method for Real-Time Biometric Tracking and Visualization
8. A method for real-time biometric tracking and visualization in live sports streaming, comprising capturing real-time biometric data from an athlete using a wearable heart rate monitor; preprocessing and filtering the biometric data within a mobile application, wherein signal noise is removed and updates are transmitted only when heart rate fluctuations exceed a predefined threshold or at a fixed time interval; transmitting the processed biometric data to a backend server, wherein the data is synchronized based on match sessions and made available via a secure API; retrieving real-time biometric data via a web-based overlay system, wherein the system dynamically adjusts graphical exertion levels in response to real-time heart rate changes; and integrating the graphical overlay with a live video stream, wherein the overlay displays exertion-based health bars, real-time BPM counters, and comparative multi-athlete biometric trends.

9. The method of claim 8, wherein multi-athlete biometric data is synchronized and displayed side by side for real-time comparative analysis.

10. The method of claim 8, wherein biometric data is transmitted using an adaptive data prioritization model that minimizes bandwidth consumption without compromising real-time updates.

11. The method of claim 8, wherein the web-based overlay system supports interactive graphical elements, allowing users to toggle between different biometric metrics, including heart rate, heart rate variability (HRV), and oxygen saturation.

12. Non-Transitory Computer-Readable Medium
12. A non-transitory computer-readable medium storing instructions that, when executed by a processor, cause a computing device to perform the method of claim 8.

13. System for Multi-User Biometric Tracking
13. A system for multi-user biometric tracking in live sports competitions, comprising a plurality of wearable heart rate monitors, each assigned to an individual athlete and configured to collect and transmit biometric data via Bluetooth, Wi-Fi, or 5G to a centralized system; a mobile application configured to pair with multiple wearable monitors, generate a shared match session ID for synchronized biometric tracking, and manage real-time biometric synchronization across multiple competitors in a single event; a backend server configured to store and synchronize match-specific biometric data to ensure real-time accessibility for overlay visualization; and a web-based overlay system configured to retrieve biometric data for multiple athletes simultaneously, display real-time comparative exertion levels using dynamically updating graphical health bars, and synchronize live graphical overlays with third-party streaming platforms.

14. The system of claim 13, wherein biometric data is displayed with dynamically updating health bars representing exertion levels relative to predefined athlete-specific thresholds.

15. The system of claim 13, wherein real-time alerts are triggered when an athlete's heart rate exceeds a predefined safety threshold, notifying coaches or sports analysts.
